(19) United States
(12) Patent Application Publication
(10) Pub. No.: US 2025/0132045 A1
(43) Pub. Date: Apr. 24, 2025

(54) A SYSTEM, COMPUTER PROGRAM AND METHOD

(71) Applicant: Sony Group Corporation, Tokyo (JP)

(72) Inventors: Ichigo Okai, Tokyo (JP); David Duffy, London (GB); Christopher Wright, London (GB)

(73) Assignee: Sony Group Corporation, Tokyo (JP)

(21) Appl. No.: 18/687,423

(22) PCT Filed: Jun. 24, 2022
(86) PCT No.: PCT/JP2022/025379

§ 371 Date: Feb. 28, 2024

(30) Foreign Application Priority Data
Sep. 10, 2021 (EP) 21196012.5

(51) Int. Cl. G16H 50/30; G10L 15/18

(52) U.S. Cl. CPC G16H 50/30; G10L 15/1807

(57) ABSTRACT

A system, comprising circuitry configured to: receive speech data associated with speaking of a user and motion data indicating head motion of the user, the motion data being captured by a device worn by the user; compare the speech data and the motion data to predict speech related motion; and determine a Schizophrenia parameter based upon the speech related motion.

### TECHNICAL FIELD

The present technique relates to a system, computer program and method.

### BACKGROUND

The “background” description provided herein is for the purpose of generally presenting the context of the disclosure. Work of the presently named inventors, to the extent it is described in the background section, as well as aspects of the description which may not otherwise qualify as prior art at the time of filing, are neither expressly or impliedly admitted as prior art against the present technique.

Research has indicated that during speech, humans make associated head motions that are driven by the rhythm or prosody of the words being spoken. The size of these speech-related head motions is a strong predictor of schizophrenia, and schizophrenia symptom severity.

In one particular study, researchers asked schizophrenia sufferers and healthy control subjects to answer a question while a video of them speaking was recorded. The size of the subject's head motions during their speech was determined via video processing, and the researchers identified a significant reduction in average head motion for users who suffered from schizophrenia.

With schizophrenia, timely assessment of changes in symptoms can have a significant impact on the quality of life of the sufferers. However, due to the complexity and intrusive nature of the systems set out in the current research regular monitoring of the sufferer is not possible. Moreover, Schizophrenia sufferers may find it difficult to monitor and objectively assess their own symptoms, making it difficult to maintain independence and control over their own condition.

“Computer Vision-Based Assessment of Motor Functioning in Schizophrenia: Use of Smartphones for Remote Measurement of Schizophrenia Symptomatology”, Anzar Abbas et al Digial Biomarkers Digit Biomark 2021; 5:29-36: DOI: 10.1159/000512383 describes prior art.

It is an aim of the disclosure to address at least one of the above issues.

### SUMMARY

Embodiments of the disclosure are defined by the appended claims.

The foregoing paragraphs have been provided by way of general introduction, and are not intended to limit the scope of the following claims. The described embodiments, together with further advantages, will be best understood by reference to the following detailed description taken in conjunction with the accompanying drawings.

### BRIEF DESCRIPTION OF DRAWINGS

A more complete appreciation of the disclosure and many of the attendant advantages thereof will be readily obtained as the same becomes better understood by reference to the following detailed description when considered in connection with the accompanying drawings.

FIG. 1 shows a head mounted device 100 and a Schizophrenia assessment apparatus 200 according to embodiments of the present disclosure.

FIG. 2 shows a system having the head mounted device 100 and the Schizophrenia assessment apparatus 200 according to embodiments of the present disclosure.

FIG. 3 shows a process explaining the structure of computer software according to embodiments of the disclosure.

FIG. 4 shows a process explaining FIG. 3 according to embodiments.

FIG. 5 shows an example of prosodic hierarchy.

FIG. 6 shows an overlay of head motion data and speech rhythm data to extract speech-related motion according to embodiments of the disclosure.

FIG. 7 shows one embodiment of privacy protection according to embodiments.

FIG. 8 shows one embodiment of privacy protection according to embodiments.

FIG. 9A shows a schematic diagram of the head mounted device 100 according to embodiments of the present disclosure.

FIG. 9B shows a schematic diagram of the schizophrenia assessment apparatus 200 according to embodiments of the present disclosure.

FIG. 10 shows a system according to embodiments of the disclosure.

### DESCRIPTION OF EMBODIMENTS

Referring now to the drawings, wherein like reference numerals designate identical or corresponding parts throughout the several views.

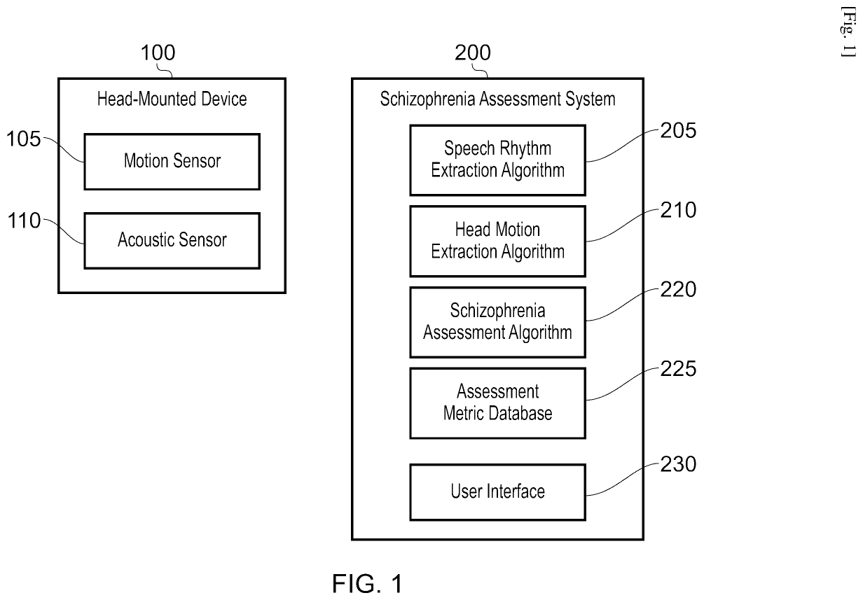

FIG. 1 shows a head mounted device 100 and a Schizophrenia assessment apparatus 200 according to embodiments of the present disclosure.

The head mounted device 100 in embodiments of the disclosure is a device that is worn by the user, typically (or thought not exclusively) on the user's head and includes a motion sensor 105 and an acoustic sensor 110. In embodiments, the head mounted device 100 may be earbuds, earphones or other “hearables”; a hearing aid; smart or augmented reality glasses; other smart devices worn on the head (e.g. hats, head- or face-mounted interfaces) or a virtual reality headset.

The acoustic sensor 110 captures the speech of the user. In some embodiments, the exact speech produced by the user may not be captured by the acoustic sensor 110. Instead, the acoustic sensor 110 may capture only the rhythm of the user's speech. The acoustic sensor 110 may take the form of a microphone, a bone-conduction vibration sensor, other sound-detecting sensors or the like.

The motion sensor 105 captures the head motion of the user whilst speaking. The motion sensor 105 may take the form of any one of an accelerometer and/or a gyroscope and may be embodied as circuitry. The motion sensor 105 is therefore able to describe natural motions that occur within the limits of the human physiology; for example, nodding/vertical motions; shaking/horizontal motions, rotational motions or the like.

The motion sensor 105 and the acoustic sensor 110 provides motion data and speech data to the Schizophrenia assessment apparatus 200. In some embodiments, the motion data and/or the speech data may be in its raw form (i.e. the user's voice is captured and simply passed to the Schizophrenia assessment apparatus 200 without anonymising). In other embodiments, the privacy of the user is assured by encrypting or otherwise anonymising the raw speech before passing to the Schizophrenia assessment apparatus 200. In other words, the speech data and/or the motion data may be in its raw form or may be anonymised prior to being passed to the Schizophrenia assessment apparatus 200. In embodiments, the speech data may be speech data that is captured when the individual is moving their head. In further embodiments, and as noted below, one or more algorithms noted as being carried out in the Schizophrenia assessment apparatus 200 is or are carried out in the head-mounted device 100 instead.

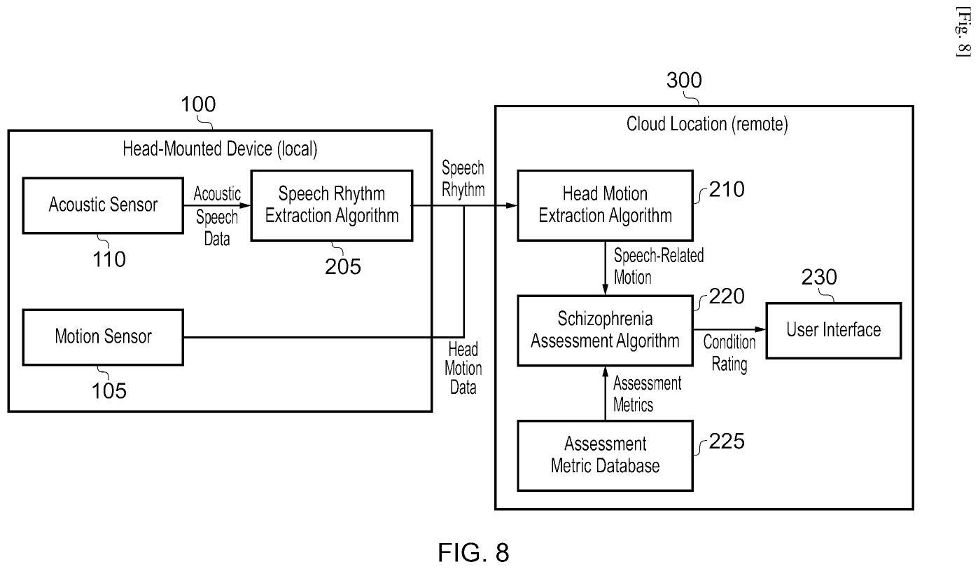

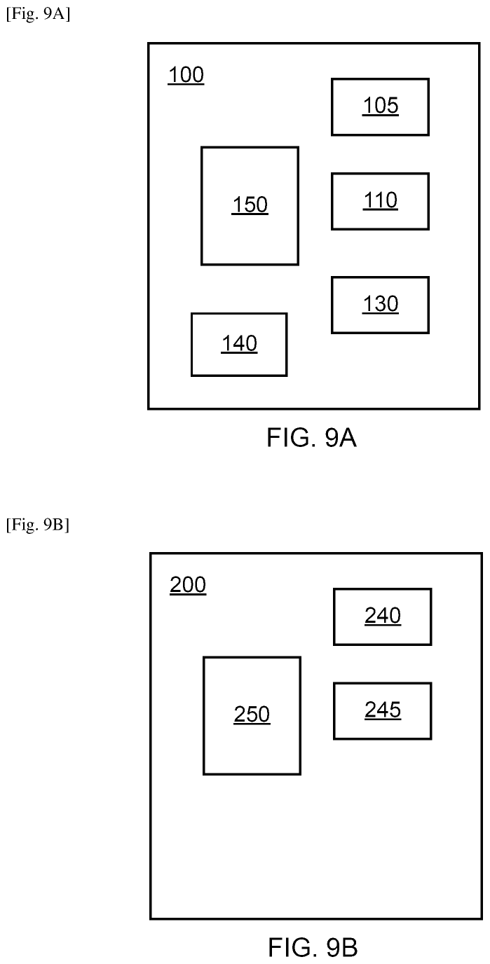

The Schizophrenia assessment apparatus 200 comprises a speech rhythm extraction algorithm 205, a head motion extraction algorithm 210, a Schizophrenia assessment algorithm 220, an assessment metric database 225 and a user interface 230. As is apparent, whilst the speech rhythm extraction algorithm 205 and/or the head motion extraction algorithm 210 are described in the following as being part of a device separate to the head mounted device 100, the disclosure is not so limited. In particular, the algorithms may be run in the head mounted device 100. Indeed, any of the algorithms may be ran on any part of a system including the head-mounted device 100 and the Schizophrenia assessment apparatus 200. This will be explained with reference to FIGS. 8 and 9.

The speech rhythm extraction algorithm 205 extracts the rhythm or prosody of a user's speech from the user's speech. This is achieved using a known technique such as that described in “An Open Source Prosodic Feature Extraction Tool”, by Huang, Zhongqiang and Chen, Lei and Harper, Mary in Proceedings of the Fifth International Conference on Language Resources and Evaluation (LREC06)”, May 2006, Genoa, Italy, European Language Resources Association (ELRA).

The head motion extraction algorithm 210 extracts head motion specifically associated with speech from the head motion data provided by the head motion sensor 105. The head motion extraction algorithm 210 uses the rhythm or prosody of the user's speech to extract the head motion data associated with the speech from the head motion provided by the head motion sensor 105. This extraction of head motion data associated with the speech is achieved by matching the rhythm or prosody of the user's speech with motion signals in the head motion data where patterns and cadence of speech (established from the prosody or rhythm of the speech) produce specific head motions that are identified in the head motion data. In other words, the prosody of the user's speech will produce certain patterns of head motion and these patterns are identified in the head motion data. This means that the rhythm of the user's speech is extracted from the received speech data. This pattern of head motion caused by the rhythm of the user's speech is speech related motion. It should be noted that whilst prosody and rhythm of the user's speech is noted here, any appropriate speech parameter is envisaged. In other words, any speech parameter that predicts the speech related motion from the speech data and the motion data is envisaged. Examples of the speech parameter includes stress applied to a word or intonation of speech.

The Schizophrenia Assessment Algorithm 220 analyses the head motion data associated with the speech extracted from the head motion provided by the head motion sensor 105 to identify the likelihood of the user displaying symptoms of Schizophrenia and/or the severity of the Schizophrenia symptoms. In other words, this likelihood and/or the severity is determined based upon the motion data associated with the rhythm of the received speech data. This likelihood and/or the severity is a Schizophrenia parameter and is sometimes termed the condition rating hereinafter.

In order to determine the condition rating, the user's head motion associated with the speech (the speech related motion) is compared to baseline metrics and/or ranges. These metrics and/or ranges may be established from existing or developing research in the field of head motion-based schizophrenia diagnosis (for example accepted metrics on head motion behaviours associated with healthy people and schizophrenia patients) and are referred to as speech related motion parameter below. In embodiments, the metrics and/or ranges may be from previously gathered head motion data associated with the speech for that particular user and percentage changes in head motion may be tracked over time which may indicate greater or lesser head motion association with the speech. In embodiments, the condition rating is determined based upon a comparison between the motion data associated with the rhythm of the received speech data of the user and an individual not having Schizophrenia.

In embodiments, the metrics may include, for example, the rate of head motion and/or the amplitude of head motion. Other metrics may include speed of speech related motion or direction of the speech related motion away from a given centre point. These metrics may be stored in an assessment metric database 225 which is accessible to the Schizophrenia Assessment Algorithm 220 and whilst shown in the Schizophrenia Assessment System 200 may instead be located on the cloud in a secure location.

In embodiments, the user interface 230 communicates the condition rating to the user or a healthcare professional. The user interface 230 may take the form of an app or notification feed on the user smartphone or the like or on a software platform or web portal accessible only by a healthcare professional.

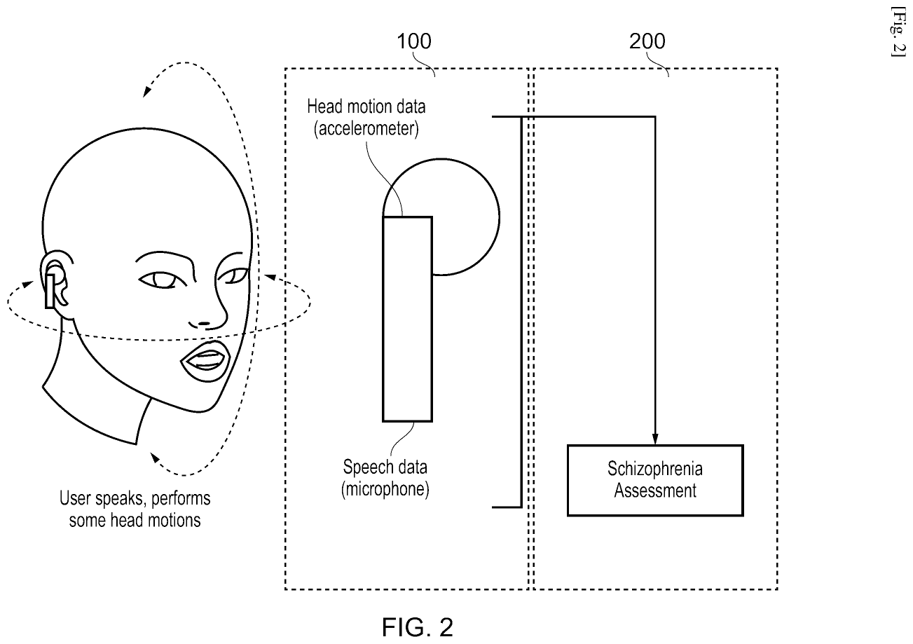

Referring to FIG. 2, a system having the head mounted device 100 and the Schizophrenia assessment apparatus 200 according to embodiments of the present disclosure is shown. In embodiments, the user wears the head mounted device 100 as he or she performs their normal activities. As can be seen, in embodiments, the head mounted device 100 is an earbud, headphone, hearing aid or similar through which the user may listen to music or amplified environmental sounds. This may be known head mounted device as existing noise cancelling headphones typically include a microphone (i.e. the acoustic sensor 100) which is used to monitor external sounds and apply appropriate noise cancelling waveforms over the user's preferred audio. Moreover, existing noise cancelling headphones also include one or more motion sensors that detect a user's head movements using accelerometers, gyroscopes and the like.

As the user speaks, the movement of the user's head is captured by the motion sensor 105 in the head-mounted device 100. In embodiments, the speech data and the motion data is sent to the Schizophrenia assessment device 200. In particular, the head motion data and the speech data is used as will be explained to generate a Schizophrenia assessment which will be presented to the individual and possibly the medical practitioner under whose care the user is.

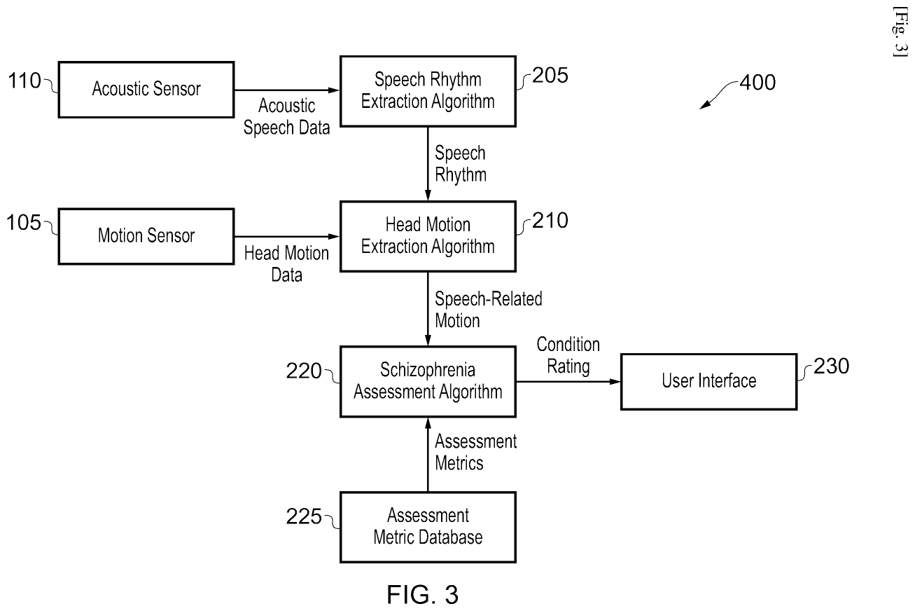

Referring to FIG. 3, a process explaining the structure of the method according to embodiments of the disclosure is shown.

The user speaks while wearing the Head-Mounted Device 100. A variety of speech events may be recorded and used with the system described. These may include one or more of the following: conversation with another person; interactions with a voice interface (e.g. a smart assistant); narration of a specific piece of text. In the example of a specific piece of text, the text may be chosen to have a specific prosody. For example, the piece of text may be a poem, song lyrics or the like. This selection of a specific piece of text having a specific prosody enables the prosody to be established more easily.

The acoustic sensor 110 detects the user's speech and captures the speech data, from which the speech rhythm extraction algorithm 205 extracts the user's speech rhythm or prosody. In embodiments, the acoustic sensor 110 may pass the user's speech (in either an unencrypted or encrypted form) to the Schizophrenia assessment device 200 without storing the speech or may instead store the speech locally.

In order to ensure that the speech rhythm extraction algorithm 205 is provided with only speech captured from the individual that should be processed and not all sounds, the acoustic sensor 110 may only capture and possibly record speech when it is determined to be from the user. This determination may be done automatically or may be done manually.

In embodiments, the acoustic sensor 110 may automatically detect when the user has begun to speak using, for example speech recognition and voice identification technologies that are known in the art. In other instances, in a system where the user wears the head-mounted device 100 on each ear, resolving the origin of a voice to the user based on the relative amplitude of the sound received by each microphone. In other words, the acoustic sensor 110 in each head-mounted device 100 is located in the user's ears and by determining the volume of the received speech in each acoustic sensor 110, the speech rhythm extraction algorithm 205 will be able to resolve if the origin of the speech was the user's mouth and begin processing the received speech and/or send a signal to the acoustic sensor 110 in each to begin capturing the speech. In other instances, if the acoustic sensor 110 is a bone conduction vibration sensor, the acoustic sensor 110 may monitor bone-conducted sound profiles via the user's head-mounted device 100 to identify that the speech has originated from the user's vocal tract only.

Of course, in embodiments, the user may manually indicate to the system that they are about to speak, which may trigger the acoustic sensor 110 to begin capturing speech. This manual indication may be achieved by, for example: allowing the user to use an app-based user interface to press a button indicating that capturing speech may begin, selecting a “wake-word” that the user may use to initiate active recording via a voice interface with the head-mounted device 100. In this example, the acoustic sensor 110 would operate via an “always on” protocol, whereby the sensor is continuously listening but not recording or transmitting what it hears until it detects that the wakeword has been spoken. However, the disclosure is not so limited. In some instances, a certain head movement sensed by the motion sensor 105 may trigger the acoustic sensor 110 to begin capturing the speech.

In instances where the acoustic sensor 110 is not “always on”, the user's interactions with the head-mounted device 100 may be used to initiate speech data recording. For example, an earbud head-mounted device 100 may include functions that allow the user to answer a call by touching a button on the side of the device. Such an action may be used as a trigger to indicate to the system that the user is about to begin speaking.

In embodiments, the speech data may be collected at a frequency, for a duration or at a time point that is acceptable to the user and/or a medical practitioner. For example, the user may manually turn the Schizophrenia assessment system 200 and the head mounted device 100 off or on depending on their environment or preferences. In instances, the Schizophrenia assessment system and the head mounted device 100 may be instructed to capture and possibly record speech data only when the user is in a specific location (e.g. at home) or at a certain time of day (e.g. outside work hours); or the Schizophrenia Assessment System 200 and the head mounted device 100 may be instructed to capture and possibly record the speech data from an entire vocal event (e.g. an entire conversation or narration), or to collect only sufficient speech data to provide an adequate assessment of head motion data associated with the speech.

In all scenarios (i.e. irrespective of the technique to commence capturing or recording of the speech), it is desirable to ensure that the speech detected is that of the user. Therefore, voice recognition technologies may be applied to any recorded speech sample to verify that only the user's speech is being analysed. Due to this, a brief calibration stage may be required, enabled via for example the user interface, to gather an initial voice sample against which further speech data may be authenticated.

The speech data may be recorded in a standard audio file format such as a .wav or .mp3 and stored and processed either locally (i.e. on a device or via a user's third party device) or remotely (i.e. transmitted to a cloud location or over a local network). The speech data may be encrypted prior to storage. This speech data is provided to the Speech Rhythm Extraction Algorithm 205.

The speech rhythm extraction algorithm 205 processes the speech data captured by the acoustic sensor 110 to obtain the rhythm and/or prosodic of the user's speech. The rhythm and/or prosodic of the user's speech is obtained by spoken features such as emphasis on certain words or phrases, identified via increases in spoken volume; change in tone or stress on the pronunciation; elongated pronunciation; temporal flow or distribution of phrasing; or semantic content or focus of the sentence. This would be appreciated by the skilled person.

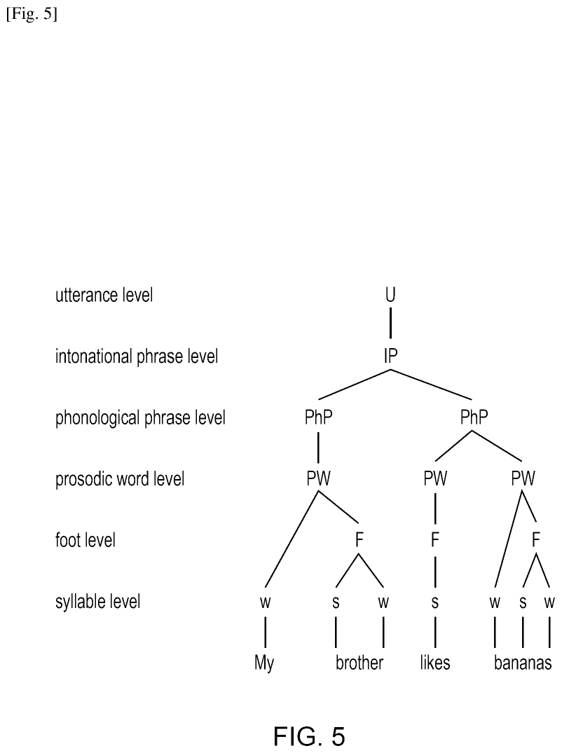

FIG. 5 shows an example of prosodic hierarchy which is used to characterise features of sentences. The similarity of those features to compositional patterns in music is used to define the location of a syllable, word or phrase in the hierarchy. Within this hierarchy, prosodic words, prominences and phrase boundaries are known to influence head motion. For example, the timing of the peak of a gesture movement is correlated with the prosodic heads and edges of the utterance and head rotation/nodding becomes maximal during prosodic events. This is a known technique and so will not be explained in any more detail.

Referring back to FIG. 3, the speech rhythm extraction algorithm 205 may therefore generate a temporal waveform (i.e. the rhythm of the user's speech) of speech features detected in the speech data that are identified as prosodic, or have audibly received emphasis in the user's speech, such that the speech features may have produced some speech-driven motion in the user's head as they were pronounced. Analysis of sentence features may be achieved using Natural Language Processing or other machine learning techniques that are known in the art.

Examples of such speech feature extraction processing are known. In one simple example, the machine learning input and output may be structured as follows: The input to the algorithmic assessment may be a complete acoustic waveform of the user's speech, showing all variations in amplitude and/or frequency over time. The algorithmic assessment output (Speech Rhythm) may vary depending on the embodiment. For example, it may consist of a set of binary “beat” values and associated timestamps (e.g. a “1” assigned for parts of the waveform where prosodic words/phrases are detected), and a waveform indicating only significant variations in amplitude/frequency over time that are associated with prosodic words and phrases.

Upon detection of speech, the motion sensor 105 is triggered to begin recording the head motion data. The head motion data is recorded simultaneously to the recording of the speech data. The head motion data is composed of one or more metric such as the speed of motion of the user's head, the direction of motion away from a given centre point (a motion vector) or the amplitude of motion (the amount of head movement from a given centre point).

In embodiments, speed of motion is measured between a first relative head position and a second relative head position. This may be defined in mm/s, where a relative head position may be indicated by a reduction of the speed metric to zero. The motion vector may be indicated by the position of the user's head in the x, y and z planes of a standard 3D frame of reference. Finally, the amplitude of motion is based on the magnitude of variation in the motion vector frame of reference.

The head motion extraction algorithm 210 uses the rhythm and/or prosody to identify the motion in the head motion data that is related to speech.

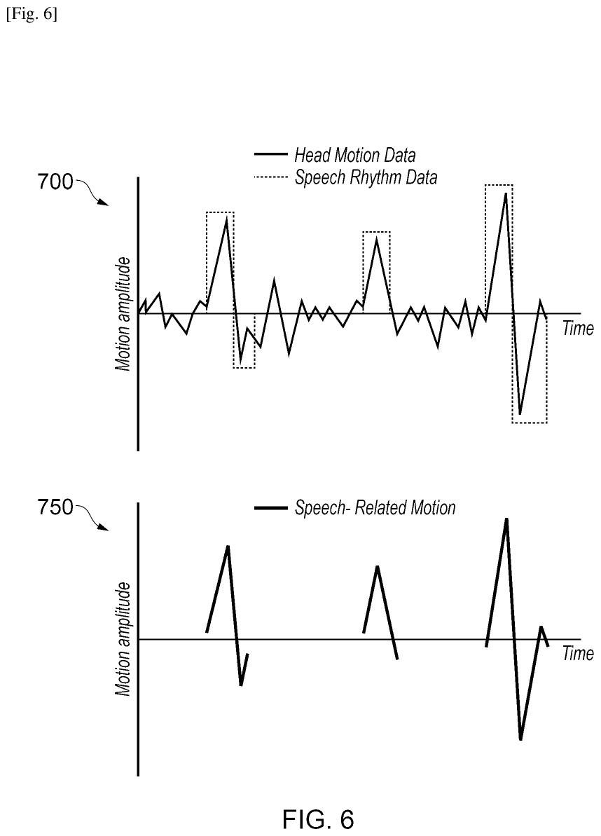

In order to do this, the temporal waveform generated by the speech rhythm extraction algorithm 205 is compared to the head motion waveform generated by the motion sensor 110 in such a way that the temporal and amplitudinal signatures of each may be matched and overlaid. This is shown graphically in FIG. 6 where the first waveform 700 shows the head motion data (the continuous waveform) and the temporal waveform generated by the speech rhythm extraction algorithm 205 (the rectangular waveform). The second waveform 750 shows the output of the head motion extraction algorithm 210. This is the head motion data associated with speech.

Speech-driven head motions associated with speech prosody/rhythm can be characterised algorithmically, where motions of the head used to add emphasis to spoken content or engage with a listener may be differentiated from non-speech head motions.

Initially, a timestamp may be applied to the speech data and head motion data such that an overlapping start point of each data set may be identified and used as the first basis for aligning the two waveforms shown in waveform 700.

Subsequently the head motion waveform (the continuous waveform in 700) may be assessed relative to the characteristic speech features present in the waveform generated by the speech extraction algorithm 205, such that:
i. The known temporal distribution of vocal features in the particular waveform generated by the speech rhythm extraction algorithm 205 may be used to identify points within the head motion data waveform where head motion data associated with speech should have occurred.
ii. The head motion data at those points may thus be defined as head motion data associated with speech and its characteristics (amplitude, speed etc.) may be recorded for later comparison with a global or user average.

The head motion data associated with speech is passed to the Schizophrenia Assessment Algorithm 220 and this is analysed to determine the user's condition rating. The condition rating is a metric used to define a Schizophrenia parameter associated with an individual. In other words, the condition rating defines how affected an individual is by Schizophrenia. This will now be explained.

Schizophrenia sufferers have been shown to exhibit a much lower rate of head movement than individuals without Schizophrenia. This will likely be correlated to symptom severity. As a result, changes in rate of head movement detected in head motion data associated with speech may be used to determine a user's condition rating.

The condition rating may be expressed as a percent reduction in head motion data associated with speech from a user average or accepted value range. For example, some research has demonstrated that individuals without Schizophrenia have an average head movement rate of 2.50 mm/frame, compared to 1.48 mm/frame for schizophrenia sufferers. This equates to a head movement percentage reduction of 41% and so in embodiments equates to a condition rating of 41%. Note that the rate of head movement is given in mm/frame in this reference as the analysis was conducted by comparing relative motion between images of the participant's head. This may be converted to units that may be comparable to head motion data captured by the motion sensor 105 (e.g. mm/s) with appropriate information on the frame rate used to capture the images.

The percentage value may additionally be associated with appropriate user warnings on increasing symptom severity (as guided by medical recommendations and accepted medical discourse). For example, given schizophrenia symptoms such as delusions, hallucinations, disorganised thinking and speech and disorganised motor behaviour:
At a 20% condition rating, the individual may experience mild occurrences of disorganised thinking and speech and disorganised motor behaviour.
At a 40% Condition Rating, the individual may experience severe occurrences of disorganised thinking and speech and disorganised motor behaviour, as well as mild symptoms of delusions and hallucinations.

Of course, an individual presenting Schizophrenia symptoms may show one or more symptoms associated with Schizophrenia with varying degrees of severity for any given condition rating. For example, an individual may have severe occurrences of disorganised thinking and no other symptoms. This may mean that they are given a condition rating of 15%. The link between the number and severity of symptoms and the condition rating will be defined by a medical practitioner and will be consistent amongst individuals under test.

In embodiments, the average head movement of individuals with varying degrees of Schizophrenia severity will be stored in the assessment metric database 225. In other words, the average head movement of a population of individuals with medically defined condition ratings that indicate low, medium and high severity Schizophrenia will be stored within the assessment metric database 225 and this population will be used to determine the severity of the Schizophrenia attributed to the individual under test. This will provide the severity of the Schizophrenia attributed to the individual under test at the time of taking the test. However, whilst very useful, this does not provide an ongoing trend associated with the individual. In other words, it is useful to establish whether the individual is getting worse symptoms or if medication is helping the individual's symptoms and the like.

Therefore, in embodiments, the user's average head motion data associated with speech may be tracked over time to identify changes in the user's condition rating. For example, lower than average head motion data associated with speech may indicate worsening symptoms in a diagnosed user, or onset of symptoms in an undiagnosed user. Average head motion data associated with speech may be gathered from the user at regular intervals and stored in the assessment metric database 225, such that a reliable measurement may be made.

For example:
One measurement of an arbitrary length may be collected each day during normal use, its head motion data associated with speech extracted and the associated motion metrics (speed, amplitude etc.) stored in the assessment metric database 225.
A measurement may be made at the request of the individual or their medical supervision if the individual is presenting with more severe symptoms or if a change in medication has been prescribed. This will reduce the risk of sudden changes to the dosage regime or pharmaceuticals negatively affecting the individual.
Moving averages of the various motion metrics may be calculated by the Schizophrenia assessment algorithm 220 as new measurements and collected over time. These may also be stored in the assessment metric database 225.
The user's most recent head motion data associated with speech values may be compared to the appropriate moving average to indicate changes in the user's motor function.
Where changes in the head motion data associated with speech are identified, a predetermined change in measurement behaviour may be triggered such that a larger number of measurements are taken to increase the accuracy of the prediction. For example, where there is a change of 2% in the condition rating over a period of time, such as one month, then the length of each measurement taken may be longer and/or the frequency of taking the measurement is increased.

In embodiments, averages for individuals identified as not having Schizophrenia may be used as a baseline comparison against which the condition rating may be determined. For example, data extracted from peer-reviewed scientific research and medical best practice may be used to indicate an appropriate threshold or value for an individual not having schizophrenia, such that deviation of a certain degree from this value may indicate symptoms of schizophrenia. Relevant assessment metrics may be stored in the assessment metric database 225, having been manually added or scraped from trusted web sources using machine learning techniques that are known in the art.

As noted above, existing research has demonstrated a rate of head movement in individuals not having Schizophrenia (measured across the x-y-z planes) of 2.50 mm/frame, compared to 1.48 mm/frame in diagnosed schizophrenia sufferers. Clearly, research into this area of diagnostic medicine is progressing and this further research, in embodiments, will be incorporated into the assessment criteria made by the Schizophrenia assessment algorithm 220 as it emerges.

In embodiments, the averages stored within the assessment metric database 225 may, where appropriate, be further subdivided by age, gender and other relevant demographic information such that a user's head motion data associated with speech may be compared to the average head motion data associated with speech of individuals in a matching demographic. This enables a more accurate diagnosis to be made.

In this regard, the user interface 230 may be configured to allow a user to enter relevant demographic information about themselves such as age, gender, medication taken and the like. In embodiments, this is used to identify the appropriate head motion data associated with speech comparison sets stored in the assessment metric database 225.

In embodiments, head motion data associated with speech may be collected from users of other head-mounted devices 100 to characterise the head motion data associated with speech of individuals not having schizophrenia. This data may then be used in a similar manner to a global or demographically subdivided head motion data associated with speech average, where data collected from individuals not having Schizophrenia may be used to determine a baseline comparison point for calculation of a specific user's condition rating.

In this regard, users not having Schizophrenia may consent to share their head motion data associated with speech for use in a wider average classification to identify individuals with Schizophrenia. In order to identify these users who do not have Schizophrenia, these individuals may confirm via the user interface 230 that they have not received a schizophrenia diagnosis from a medical professional, allowing their data to be used in the calculation of, say, an upper threshold for head motion data associated with speech for users not having Schizophrenia. Similarly, users with a confirmed diagnosis may share this information to allow a threshold for head motion data associated with speech that is symptomatic of schizophrenia. This data may be anonymised before being stored.

The condition rating produced by the Schizophrenia assessment algorithm 220 is communicated to the user via the user interface 230. A threshold may be set, arbitrarily or based on approved medical practice, at which changes in the user's condition rating may be indicated to the user or medical practitioner. For example, notifications might be shared when there is an extreme reduction in head motion data associated with speech detected. In other words, when the condition of the individual deteriorates more than a predetermined threshold, the individual and/or the medical practitioner in whose care the individual is will be informed. In embodiments, notifications might also be shared where the individual's head motion data associated with speech is observed to remain consistently reduced over a certain number of days. In embodiments, a regular report summarising the user's condition rating over one or more days may be shared with the individual via an app interface or similar.

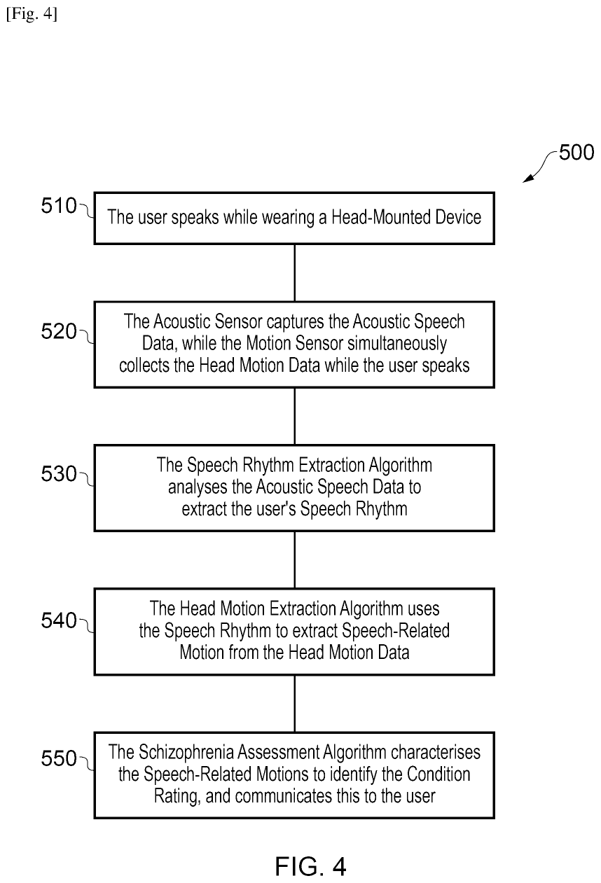

Referring to FIG. 4, a process explaining FIG. 3 according to embodiments is described.

The process 500 starts at step 510 where the user speaks whilst wearing the head-mounted device 100. As noted above, the acoustic sensor 110 in the head-mounted device 100 will detect that the user is speaking (either automatically or by the individual manually indicating that they are speaking) and will capture the audio when speech is detected.

The process moves to step 520 where the acoustic sensor 110 captures the speech data and the motion sensor 105 simultaneously captures the head motion data whilst the individual speaks. The process moves to step 530 where the speech rhythm extraction algorithm 205 analyses the speech data captured by the audio sensor 110 to extract the user's speech rhythm or prosody. The process then moves to step 540 where the extracted speech rhythm or prosody is used to determine the head movement captured by the motion sensor 105 that corresponds to the extracted speech rhythm or prosody. This determines the head motion due to speech.

The process then moves to step 550 where the Schizophrenia assessment algorithm 220 determines the condition rating from the determined head motion due to speech. The condition rating is then output to a user interface.

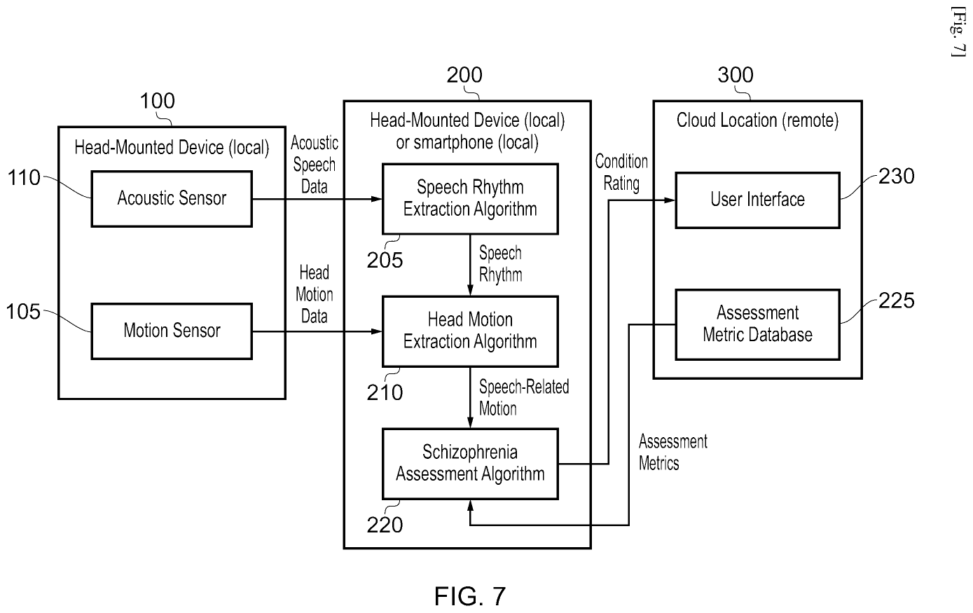

As discussed with reference to FIG. 1, in embodiments, the head-mounted device 100 includes the motion sensor 105 and the acoustic sensor 110. Also in FIG. 1, the Schizophrenia Assessment device 200 runs the speech rhythm extraction algorithm 205, the head motion extraction algorithm 210, the Schizophrenia assessment algorithm 220, the assessment metric database 225 and the user interface 230. However, the disclosure is not so limited and any of these functions may be performed in any part of the system of FIG. 1 or in embodiments, may involve devices located on the cloud. This will now be explained with reference to FIGS. 7 and 8. This will, in embodiments, result in increased privacy for the user.

In FIG. 7, the head mounted device 100 includes the acoustic sensor 110 and the motion sensor 105 as in FIG. 1. However, the speech rhythm extraction algorithm 205, the head motion extraction algorithm 210, the Schizophrenia assessment algorithm 220 is run in either the head mounted device 100 or a user's smartphone, tablet or PC which is connected to the head mounted device 100. This connection may be via a secure Bluetooth connection or the like.

The output from the Schizophrenia assessment algorithm 220 (the condition rating) may be fed to a user interface 230 located on a network such as over the Internet (i.e. in a location that is remote from the user). This may allow a user to access the results over the Internet. Moreover, the content of the assessment metric database 225 may be located remotely. This allows a provider to limit access to this database and to control its distribution closely which improves security. The assessment metrics used to determine the condition rating are then provided to the Schizophrenia assessment algorithm 220 when required. Moreover, in this configuration, as the speech related motion is extracted on the user's device(s), the user's personal data does not leave their device(s).

In FIG. 8, the head mounted device 100 includes the acoustic sensor 110 and the motion sensor 105 and the speech rhythm extraction algorithm 205. This means that the output of the head mounted device 100 is the speech rhythm and the head motion data. This is fed to the head motion extraction algorithm 210 that is located in a cloud location 300 such as over the internet (i.e. in a location remote to the user). Also provided in the remote location is the Schizophrenia assessment algorithm 220, the assessment metric database 225 and the user interface 230.

In this case, the Acoustic Speech Data is extracted locally, without being transferred to the cloud location. Only the Speech Rhythm associated with the Acoustic Speech Data is transmitted to the cloud location, not the actual speech data. In order for the Speech-Related Motion to be extracted at the cloud location, the Head Motion Data should also be transferred to the cloud location. This again improves the privacy of the user's data.

In either scenario, the user's speech data is protected. As such, the preference for either scenario may be determined relative to the processing requirements of the various algorithmic components and the capabilities of the head-mounted device 100 and personal device to conduct them.

FIGS. 9A and 9B shows a schematic diagram of the head mounted device 100 and the Schizophrenia assessment apparatus 200 according to embodiments of the present disclosure.

The head mounted device 100 comprises circuitry that performs the various functions defined above. The is head mounted device processing circuitry 150 which is embodied in semiconductor. An example of such head mounted device processing circuitry 150 is an application specific integrated circuit (ASIC) 150 or any kind of circuitry that is configured by software to control the various parts of the head mounted device 100 to perform the functions defined above.

Additionally, provided in the head mounted device is the motion sensor 105 and the acoustic sensor 110 as noted above. Further, head mounted device storage 130 is provided. This may be solid state or magnetically readable storage media that is configured to store computer software therein or thereon. The computer software is provided to control the head mounted processing circuitry 150 noted above.

Finally, display and communication circuitry 140 is provided in the head mounted device 100. The display and communication circuitry 140 provides a screen which displays the graphical user interface. This screen may be a touch screen. Moreover, the display and communication circuitry 140 communicates with the Schizophrenia assessment apparatus 200 either wirelessly or over a wired connection such as via Bluetooth, WiFi, Ethernet or the like.

The Schizophrenia assessment apparatus 200 comprises circuitry that performs the various functions defined above. The is Schizophrenia assessment processing circuitry 550 which is embodied in semiconductor. An example of such Schizophrenia assessment processing circuitry 550 is an application specific integrated circuit (ASIC) or any kind of circuitry that is configured by software to control the various parts of the Schizophrenia assessment apparatus 200 to perform the functions defined above.

Schizophrenia assessment apparatus storage 240 is provided. This may be solid state or magnetically readable storage media that is configured to store computer software therein or thereon. The computer software is provided to control the Schizophrenia assessment apparatus processing circuitry 250 noted above.

Communication circuitry 245 is also provided in the Schizophrenia assessment apparatus 200. The communication circuitry 245 communicates with the head mounted device 100 either wirelessly or over a wired connection such as via Bluetooth, WiFi, Ethernet or the like.

Although the above describes a system where head motion is analysed to determine the severity of schizophrenia, the disclosure is not so limited.

Diagnosed schizophrenics are known to suffer from noise sensitivity. A head mounted device 100 equipped with noise cancelling technologies may therefore be used to assist a user with this symptom, based on their determined condition rating.

In particular, where the condition rating indicates that the user is experiencing a schizophrenic episode, a noise cancelling profile whose degree of noise cancelling is associated with the condition rating. So, for example, the noise cancelling profile will simply turn on maximum noise cancelling when a particular condition rating threshold is reached. Alternatively, where it can be said that the level of noise sensitivity increases with the severity of the schizophrenia episode, the profile may be designed to adjust the level of noise cancelling applied with increasing condition rating.

The user experiences the noise cancelling profile through their head mounted device 100. The response of the user to the noise cancelling profile may be monitored through using the technique described above where a new condition rating may be calculated to assess whether the noise cancelling profile has altered the user's symptoms.

Conditions such as dementia and depression are known to affect speech, and research has indicated that these conditions can be detected in features of speech. Symptoms may include a slower rate of speech, reduced pitch variability, and more pauses. As such, the present disclosure may be applied to the detection and monitoring of such additional conditions. Head motion during speech may not be required to assess other conditions, therefore related components in the system may be excluded in this embodiment.

The user's speech is extracted using the head mounted device 100 as described above, and processed by the speech rhythm extraction algorithm 205 to detect appropriate speech features for the condition being assessed. An algorithm analogous to the Schizophrenia assessment algorithm 220 applies appropriate metrics to the speech rhythm to detect features of the relevant condition (such as reduced speed of speech, pitch variability etc.) and applies a condition rating in a similar manner. As for the embodiment described above, the Condition Rating may be based on appropriate medical or personal averages or thresholds. This condition rating is then used as described above to inform the user or an appropriate medical professional about the user's health.

Although the foregoing explains using a head-mounted device 100, the disclosure is not so limited. The user may wear the device on any part of their body; the only requirement is that the head motion of the user is captured. For example, a user may wear a chest mounted camera that captures the movement of the user's head using video.

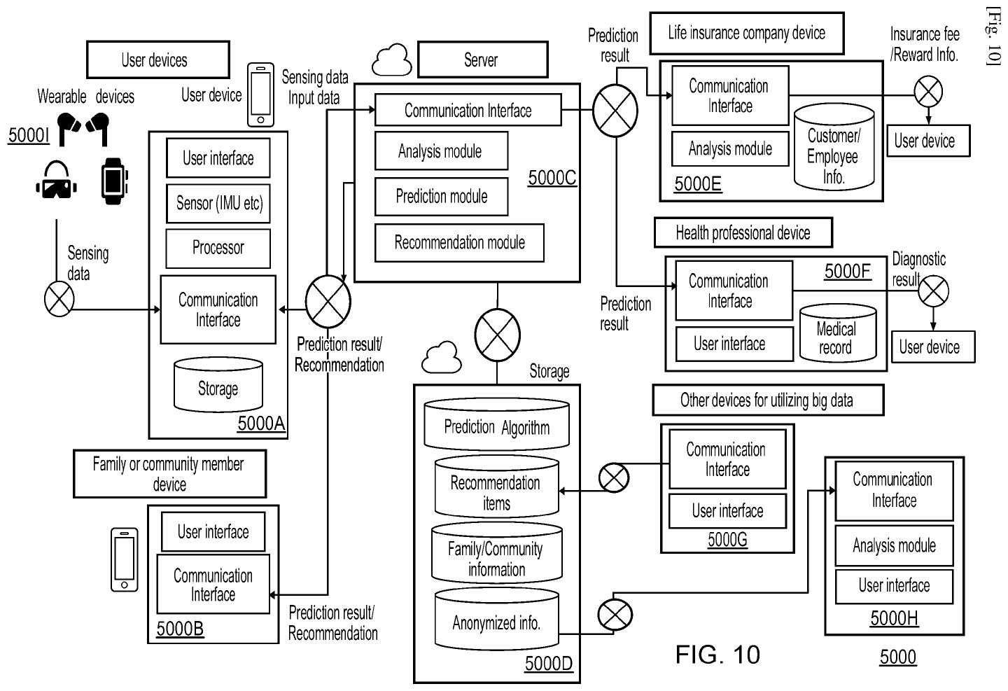

Although the foregoing has been described with reference to embodiments being carried out on a device or various devices, the disclosure is not so limited. In embodiments, the disclosure may be carried out on a system 5000 such as that shown in FIG. 10.

In the system 5000, the wearable devices 50001 are devices that are worn on a user's body. For example, the wearable devices may be earphones, a smart watch, Virtual Reality Headset or the like. The wearable devices contain sensors that measure the movement of the user and which create sensing data to define the movement or position of the user. This sensing data is provided over a wired or wireless connection to a user device 5000A. Of course, the disclosure is not so limited. In embodiments, the sensing data may be provided directly over an internet connection to a remote device such as a server 5000C located on the cloud. In further embodiments, the sensing data may be provided to the user device 5000A and the user device 5000A may provide this sensing data to the server 5000C after processing the sensing data.

In the embodiments shown in FIG. 10, the sensing data is provided to a communication interface within the user device 5000A. The communication interface may communicate with the wearable device(s) using a wireless protocol such as low power Bluetooth or WiFi or the like.

The user device 5000A is, in embodiments, a mobile phone or tablet computer. The user device 5000A has a user interface which displays information and icons to the user. Within the user device 5000A are various sensors such as gyroscopes and accelerometers that measure the position and movement of a user. The operation of the user device 5000A is controlled by a processor which itself is controlled by computer software that is stored on storage. Other user specific information such as profile information is stored within the storage for use within the user device 5000A. As noted above, the user device 5000A also includes a communication interface that is configured to, in embodiments, communicate with the wearable devices. Moreover, the communication interface is configured to communicate with the server 5000C over a network such as the Internet. In embodiments, the user device 5000A is also configured to communicate with a further device 5000B. This further device 5000B may be owned or operated by a family member or a community member such as a carer for the user or a medical practitioner or the like. This is especially the case where the user device 5000A is configured to provide a prediction result and/or recommendation for the user. The disclosure is not so limited and in embodiments, the prediction result and/or recommendation for the user may be provided by the server 5000C.

The further device 5000B has a user interface that allows the family member or the community member to view the information or icons. In embodiments, this user interface may provide information relating to the user of the user device 5000B such as diagnosis, recommendation information or a prediction result for the user. This information relating to the user of the user device 5000B is provided to the further device 5000B via the communication interface and is provided in embodiments from the server 5000C or the user device 5000A or a combination of the server 5000C and the user device 5000A.

The user device 5000A and/or the further device 5000B are connected to the server 5000C. In particular, the user device 5000A and/or the further device 5000B are connected to a communication interface within the server 5000C. The sensing data provided from the wearable devices and or the user device 5000A are provided to the server 5000C. Other input data such as user information or demographic data is also provided to the server 5000C. The sensing data is, in embodiments, provided to an analysis module which analyses the sensing data and/or the input data. This analysed sensing data is provided to a prediction module that predicts the likelihood of the user of the user device having a condition now or in the future and in some instances, the severity of the condition. The predicted likelihood is provided to a recommendation module that provides a recommendation to the user and/or the family or community member. Although the prediction module is described as providing the predicted likelihood to the recommendation module, the disclosure is not so limited and the predicted likelihood may be provided directly to the user device 5000A and/or the further device 5000B.

Additionally, connected to or in communication with the server 5000C is storage 5000D. The storage 5000D provides the prediction algorithm that is used by the prediction module within the server 5000C to generate the predicted likelihood. Moreover, the storage 5000D includes recommendation items that are used by the recommendation module to generate the recommendation to the user. The storage 5000D also includes in embodiments family and/or community information. The family and/or community information provides information pertaining to the family and/or community member such as contact information for the further device 5000B.

Also provided in the storage 5000D is an anonymised information algorithm that anonymises the sensing data. This ensures that any sensitive data associated with the user of the user device 5000A is anonymised for security. The anonymised sensing data is provided to one or more other devices which is exemplified in FIG. 10 by device 5000H. This anonymised data is sent to the other device 5000H via a communication interface located within the other device 5000H. The anonymised data is analysed with the other data 5000H by an analysis module to determine any patterns from a large number set of sensing data. This analysis will improve the recommendations made by the recommendations module and will improve the predictions made from the sensing data. Similarly, a second other device 5000G is provided that communicates with the storage 5000D using a communication interface.

Returning now to server 5000C, as noted above, the prediction result and/or the recommendation generated by the server 5000C is sent to the user device 5000A and/or the further device 5000B.

Although the prediction result is used in embodiments to assist the user or his or her family member or community member, the prediction result may be also used to provide more accurate health assessments for the user. This will assist in purchasing products such as life or health insurance or will assist a health professional. This will now be explained.

The prediction result generated by server 5000C is sent to the life insurance company device 5000E and/or a health professional device 5000F. The prediction result is passed to a communication interface provided in the life insurance company device 5000E and/or a communication interface provided in the health professional device 5000F. In the event that the prediction result is sent to the life insurance company device 5000E, an analysis module is used in conjunction with the customer information such as demographic information to establish an appropriate premium for the user. In instances, rather than a life insurance company, the device 5000E could be a company's human resources department and the prediction result may be used to assess the health of the employee. In this case, the analysis module may be used to provide a reward to the employee if they achieve certain health parameters. For example, if the user has a lower prediction of ill health, they may receive a financial bonus. This reward incentivises healthy living. Information relating to the insurance premium or the reward is passed to the user device.

In the event that the prediction result is passed to the health professional device 5000F, a communication interface within the health professional device 5000F receives the prediction result. The prediction result is compared with the medical record of the user stored within the health professional device 5000F and a diagnostic result is generated. The diagnostic result provides the user with a diagnosis of a medical condition determined based on the user's medical record and the diagnostic result is sent to the user device.

Numerous modifications and variations of the present disclosure are possible in light of the above teachings. It is therefore to be understood that within the scope of the appended claims, the disclosure may be practiced otherwise than as specifically described herein.

In so far as embodiments of the disclosure have been described as being implemented, at least in part, by software-controlled data processing apparatus, it will be appreciated that a non-transitory machine-readable medium carrying such software, such as an optical disk, a magnetic disk, semiconductor memory or the like, is also considered to represent an embodiment of the present disclosure.

It will be appreciated that the above description for clarity has described embodiments with reference to different functional units, circuitry and/or processors. However, it will be apparent that any suitable distribution of functionality between different functional units, circuitry and/or processors may be used without detracting from the embodiments.

Described embodiments may be implemented in any suitable form including hardware, software, firmware or any combination of these. Described embodiments may optionally be implemented at least partly as computer software running on one or more data processors and/or digital signal processors. The elements and components of any embodiment may be physically, functionally and logically implemented in any suitable way. Indeed the functionality may be implemented in a single unit, in a plurality of units or as part of other functional units. As such, the disclosed embodiments may be implemented in a single unit or may be physically and functionally distributed between different units, circuitry and/or processors.

Although the present disclosure has been described in connection with some embodiments, it is not intended to be limited to the specific form set forth herein. Additionally, although a feature may appear to be described in connection with particular embodiments, one skilled in the art would recognize that various features of the described embodiments may be combined in any manner suitable to implement the technique.

Embodiments of the present technique can generally be described by the following numbered clauses:

(1)

A system, comprising circuitry configured to:
receive speech data associated with speaking of a user and motion data indicating head motion of the user, the motion data being captured by a device worn by the user;
compare the speech data and the motion data to predict speech related motion;
and
determine a Schizophrenia parameter based upon the speech related motion.
   (2)

A system according to (1), wherein the circuitry is configured to:
detect the user is speaking; and
capture the motion data in response to detecting that the user is speaking.
   (3)

A system according to (2), wherein the circuitry is configured to:
capture the speech data in response to detecting that the user is speaking.
   (4)

A system according to (2) or (3), wherein detecting the user is speaking is based on at least one of the speech data, manual input data, and sensing data.

(5)

A system according to any preceding clause, wherein the circuitry is configured to:
determine speech parameter from the received speech data;
compare the speech data and the motion data based on the speech parameter to predict speech related motion; and
determine the Schizophrenia parameter based upon the speech related motion.
   (6)

A system according to (5), wherein the speech parameter includes at least one of speech rhythm and prosody.

(7)

A system according to any preceding clause, wherein the Schizophrenia parameter indicates likelihood and/or severity of the Schizophrenia symptoms.

(8)

A system according to (7), wherein the circuitry is configured to:
determine speech related motion parameter based on the speech related motion; and determine condition rating of the speech related motion as the Schizophrenia parameter.
   (9)

A system according to (8), wherein the speech related motion parameter includes at least one of speed of speech related motion, direction of the speech related motion away from a given centre point and amplitude of the speech related motion.

(10)

A system according to (8) or (9), wherein the condition rating is determined based upon a comparison between the speech related motion of the user and an individual not having Schizophrenia.

(11)

A system according to (10), wherein the condition rating is determined based upon a comparison with user's average of the speech related motion.

(12)

A system according to any preceding clause, wherein the circuitry is configured to:
provide the Schizophrenia parameter to either the user or a medical practitioner.
   (13)

A system according to any preceding clause configured as a head-mounted device.

(14)

A system according to (13), wherein the head-mounted device is either a headphone or a hearing aid.

(15)

A method, comprising:
receiving speech data associated with speaking of a user and motion data indicating head motion of the user, the motion data being captured by a device worn by the user;
comparing the speech data and the motion data to predict speech related motion;
and
determining a Schizophrenia parameter based upon the speech related motion.
   (16)

A method according to (15), comprising:
detecting the user is speaking; and
capturing the motion data in response to detecting that the user is speaking.
   (17)

A method according to (16), comprising:
capturing the speech data in response to detecting that the user is speaking.
   (18)

A method according to (16) or (17), wherein detecting the user is speaking is based on at least one of the speech data, manual input data, and sensing data.

(19)

A method according to any one of (15) to (18), comprising:
determining speech parameter from the received speech data;
comparing the speech data and the motion data based on the speech parameter to predict speech related motion; and
determining the Schizophrenia parameter based upon the speech related motion.
   (20)

A method according to (19), wherein the speech parameter includes at least one of speech rhythm and prosody.

(21)

A method according to any one of (15) to (20), wherein the Schizophrenia parameter indicates likelihood and/or severity of the Schizophrenia symptoms.

(22)

A method according to (21), comprising:
determining speech related motion parameter based on the speech related motion; and
determining condition rating of the speech related motion as the Schizophrenia parameter.
   (23)

A method according to (22), wherein the speech related motion parameter includes at least one of speed of speech related motion, direction of the speech related motion away from a given centre point and amplitude of the speech related motion.

(24)

A method according to (22) or (23), wherein the condition rating is determined based upon a comparison between the speech related motion of the user and an individual not having Schizophrenia.

(25)

A method according to (24), wherein the condition rating is determined based upon a comparison with user's average of the speech related motion.

(26)

A method according to any one of (15) to (25), comprising:
providing the Schizophrenia parameter to either the user or a medical practitioner.
   (27)

A method according to any one of (15) to (26), wherein the device is a head-mounted device.

(28)

A method according to (27), wherein the head-mounted device is either a headphone or a hearing aid.

1. A system, comprising circuitry configured to:
receive speech data associated with speaking of a user and motion data indicating head motion of the user, the motion data being captured by a device worn by the user;
compare the speech data and the motion data to predict speech related motion;
and
determine a Schizophrenia parameter based upon the speech related motion.

2. A system according to claim 1, wherein the circuitry is configured to:
detect the user is speaking; and
capture the motion data in response to detecting that the user is speaking.

3. A system according to claim 2, wherein the circuitry is configured to:
capture the speech data in response to detecting that the user is speaking.

4. A system according to claim 2, wherein detecting the user is speaking is based on at least one of the speech data, manual input data, and sensing data.

5. A system according to claim 1, wherein the circuitry is configured to:
determine speech parameter from the received speech data;
compare the speech data and the motion data based on the speech parameter to predict speech related motion; and
determine the Schizophrenia parameter based upon the speech related motion.

6. A system according to claim 5, wherein the speech parameter includes at least one of speech rhythm and prosody.

7. A system according to claim 1, wherein the Schizophrenia parameter indicates likelihood and/or severity of the Schizophrenia symptoms.

8. A system according to claim 7, wherein the circuitry is configured to:
determine speech related motion parameter based on the speech related motion; and
determine condition rating of the speech related motion as the Schizophrenia parameter.

9. A system according to claim 8, wherein the speech related motion parameter includes at least one of speed of speech related motion, direction of the speech related motion away from a given centre point and amplitude of the speech related motion.

10. A system according to either claim 8, wherein the condition rating is determined based upon a comparison between the speech related motion of the user and an individual not having Schizophrenia.

11. A system according to claim 10, wherein the condition rating is determined based upon a comparison with user's average of the speech related motion.

12. A system according to claim 1, wherein the circuitry is configured to:
provide the Schizophrenia parameter to either the user or a medical practitioner.

13. A system according to claim 1 configured as a head-mounted device.

14. A system according to claim 13, wherein the head-mounted device is either a headphone or a hearing aid.

15. A method, comprising:
receiving speech data associated with speaking of a user and motion data indicating head motion of the user, the motion data being captured by a device worn by the user;
comparing the speech data and the motion data to predict speech related motion;
and
determining a Schizophrenia parameter based upon the speech related motion.

16. A method according to claim 15, comprising:
detecting the user is speaking; and
capturing the motion data in response to detecting that the user is speaking.

17. A method according to claim 16, comprising:
capturing the speech data in response to detecting that the user is speaking.

18. A method according to claim 16, wherein detecting the user is speaking is based on at least one of the speech data, manual input data, and sensing data.

19. A method according to claim 15, comprising:
determining speech parameter from the received speech data;
comparing the speech data and the motion data based on the speech parameter to predict speech related motion; and
determining the Schizophrenia parameter based upon the speech related motion.

20. A method according to claim 19, wherein the speech parameter includes at least one of speech rhythm and prosody.

21. A method according to claim 15, wherein the Schizophrenia parameter indicates likelihood and/or severity of the Schizophrenia symptoms.

22. A method according to claim 21, comprising:
determining speech related motion parameter based on the speech related motion; and
determining condition rating of the speech related motion as the Schizophrenia parameter.

23. A method according to claim 22, wherein the speech related motion parameter includes at least one of speed of speech related motion, direction of the speech related motion away from a given centre point and amplitude of the speech related motion.

24. A method according to claim 22, wherein the condition rating is determined based upon a comparison between the speech related motion of the user and an individual not having Schizophrenia.

25. A method according to claim 24, wherein the condition rating is determined based upon a comparison with user's average of the speech related motion.

26. A method according to claim 15, comprising:
providing the Schizophrenia parameter to either the user or a medical practitioner.

27. A method according to claim 15, wherein the device is a head-mounted device.

28. A method according to claim 27, wherein the head-mounted device is either a headphone or a hearing aid.
